(19) United States
(12) Patent Application Publication
(10) Pub. No.: US 2019/0111247 A1
(43) Pub. Date: Apr. 18, 2019

(54) DEFIBRILLATOR PADDLES WITH USE INDICATORS

(71) Applicant: KONINKLIJKE PHILIPS N.V., EINDHOVEN (NL)

(72) Inventors: MIN ZHAO, Acton, MA (US); RINUAN YE, Shenzhen (CN)

(21) Appl. No.: 16/090,278

(22) PCT Filed: Mar. 29, 2017
(86) PCT No.: PCT/EP2017/057449

§ 371 Date: Oct. 1, 2018

Related U.S. Application Data
(60) Provisional application No. 62/315,878, filed on Mar. 31, 2016.

(51) Int. Cl. A61N 1/04; A61N 1/39

(52) U.S. Cl. CPC A61N 1/046; A61N 1/3925; A61N 1/3968; A61N 1/3904; A61N 1/3993

(57) ABSTRACT

Systems and methods for enabling defibrillator paddle assembly. The defibrillator paddle assembly of the present disclosure includes an apex paddle and a sternum paddle. The method of the present disclosure includes: positioning the apex paddle on a patient's apex; positioning the sternum paddle on a patient's sternum; determining via a contact indicator a degree of contact between the patient's skin and the defibrillator paddle assembly; charging via a charge button the defibrillator paddle assembly; and delivering a shock via one or more shock buttons on the patient's body for treating the patient's cardiac arrest.

### BACKGROUND

Ventricular fibrillation and cardiac arrhythmia are life-threatening heart conditions that can result in cardiac arrest. Aside from cardiopulmonary resuscitation (CPR), one of the most effective ways to return the heart to its normal functioning state is delivering a sudden powerful electric shock on a person's body using a defibrillator. Timely defibrillation improves the chances of reestablishing the heart's normal contraction rhythms.

U.S. Pat. App. No. 2014/0005737 discloses a numbering scheme to help guide the user in the proper procedure for using a defibrillator. The procedure includes selecting an amount of energy, charging the circuit, and delivering an electrical shock to the patient. G.B. Pat. App. No. 2085593 discloses a system in which an alarm sets off when there is poor electrical contact between the defibrillator paddles and the patient's skin. The system also alerts a user via an indicator light on the defibrillator's control panel when a threshold value is exceeded.

### BRIEF SUMMARY OF THE INVENTIONS

The present disclosure relates to systems and methods for treating a patient experiencing heart inactivity such as cardiac arrest. The system of the present disclosure comprises a defibrillator paddle assembly, which includes an apex paddle, a sternum paddle, and a contact indicator. The contact indicator emits varying intensities of light depending on the degree of contact between the patient's skin and the defibrillator paddle assembly. Also, both the apex paddle and sternum paddle have adjustable size electrodes for use with patients having different body builds, such as adult and infant/child. The present disclosure also relates to systems for treating a patient experiencing heart inactivity such as cardiac arrest, wherein the system comprises a defibrillator paddle assembly made up of an apex paddle and a sternum paddle for delivering an electric current passing through the patient's heart to revive the heart's normal functions.

The method of the present disclosure comprises placing both the apex paddle and the sternum paddle to a patient's body based on instructions provided by an apex label and a sternum label. One or more sequence labels (“1”, “2” & “3”) are placed on both the apex paddle and the sternum paddle to indicate a sequence of steps for using the defibrillator paddle assembly.

### BRIEF DESCRIPTION OF THE DRAWINGS

The accompanying drawings, which are included to provide a further understanding of the inventions of the present disclosure, are incorporated herein to illustrate embodiments of the inventions of the present disclosure. Along with the description, they also serve to explain the inventive principles of the present disclosure. In the drawings:

FIG. 1 illustrates a defibrillator paddle assembly in accordance with the inventive principles of the present disclosure for use in a patient's cardiac arrest treatment.

FIG. 2 illustrates an exemplary flowchart for enabling a defibrillator paddle assembly used in a patient's cardiac arrest treatment in accordance with the inventive principles of the present disclosure.

FIG. 3 illustrates an exemplary flowchart for implementing a deactivator to prevent accidental shock to patient in accordance with the inventive principles of the present disclosure.

FIGS. 4A-4C illustrate exemplary embodiments of contact indicator and markers in accordance with the inventive principles of the present disclosure.

FIG. 5 illustrates an exemplary embodiment of labels showing the correct placement of defibrillator paddle assembly unto a patient's body in accordance with the inventive principles of the present disclosure.

FIG. 6 illustrates an exemplary embodiment of a defibrillator paddle assembly in accordance with the inventive principles of the present disclosure.

FIG. 7 illustrates an exemplary disconnection of an adult electrode from an infant/child electrode in accordance with the inventive principles of the present disclosure.

FIG. 8 illustrates an exemplary embodiment of a defibrillator in accordance with the inventive principles of the present disclosure.

### DETAILED DESCRIPTION OF EXEMPLARY EMBODIMENTS

The following are definitions of terms as used in the various embodiments of the present disclosure.

The term “defibrillator” as used herein refers to an electronic device that delivers an electric shock to the heart using electrodes for reviving a heart experiencing, for example, ventricular fibrillation.

The term “paddle” as used herein refers to an accessory which is connected to defibrillator through a port to thereby deliver energy to a patient via electrodes.

The term “electrode” as used herein refers to an electrical conductor used to establish contact with a patient's skin to allow delivery of electric shock to a patient or other impedance, such as may be structured, configured and used for testing a device, training, simulation, and/or demonstration.

The term “apex” as used herein refers to a part of the body located just below and on the left side of the pectoral muscle.

The term “sternum” as used herein refers to a part of the body located below the clavicle on the patient's upper right torso.

The term “adult electrode” as used herein refers to a part of the defibrillator paddle assembly for delivering electric shock primarily to adults and secondarily to children having bodily dimensions of an adult.

The term “infant/child electrode” as used herein refers to a part of the defibrillator paddle assembly for delivering electric shock primarily to infants/child and secondarily to adults having bodily dimensions of a child.

The term “sequence label” as used herein refers to a label such as indicators or markers used to indicate a sequence of steps for using the defibrillator paddle assembly (“1”, “2” & “3”), for example.

The term “apex label” as used herein refers to a label such as indicators or markers used to indicate the apex part of the body.

The term “sternum label” as used herein refers to a label such as indicators or markers used to indicate the sternum part of the body.

The term “contact indicator” as used herein refers to an indicator such as LED and other display device used to indicate a degree of impedance of the paddles when held against the patient's chest. This represents a degree of contact between the patient's skin and the paddles of the defibrillator paddle assembly.

The term “low state” as used herein refers to a state of the defibrillator paddle assembly wherein the defibrillator paddles and the patient's skin have poor contact or connectivity.

The term “high state” as used herein refers to a state of the defibrillator paddle assembly wherein the defibrillator paddles and the patient's skin have good contact or connectivity.

The term “deactivator” as used herein refers to an electronic component or circuit used to control the activation and deactivation of a charge button and one or more shock buttons for preventing premature shock delivery to the patient or other impedance.

The term “moisture sensor” as used herein refers to a type of sensor that determines moisture or a wetness state of the surrounding.

The present disclosure relates to a defibrillator paddle assembly comprising: an apex paddle and a sternum paddle for delivering an electric shock passing through a patient's heart; an apex label on the apex paddle and a sternum label on the sternum paddle serving as markers for accurately placing the apex paddle and the sternum paddle on the patient's body; one or more sequence labels located on both the apex paddle and the sternum paddle indicating a sequence of steps for using the defibrillator paddle assembly; a contact indicator located on the apex paddle and/or the sternum paddle; and a removable adult electrode that can be connected to or disconnected from the infant/child electrode.

The present disclosure also relates to a method for enabling a defibrillator paddle assembly to be used in treating cardiac arrest comprising: positioning an apex paddle on a patient's apex based on instructions provided by an apex label; positioning a sternum paddle on a patient's sternum based on instructions provided by a sternum label; determining via a contact indicator the degree of contact between the patient's skin and the defibrillator paddle assembly; charging via a charge button the defibrillator paddle assembly; and delivering electrical shock via one or more shock buttons to the patient's body for treating, for example, cardiac dysrhythmias or ventricular fibrillation.

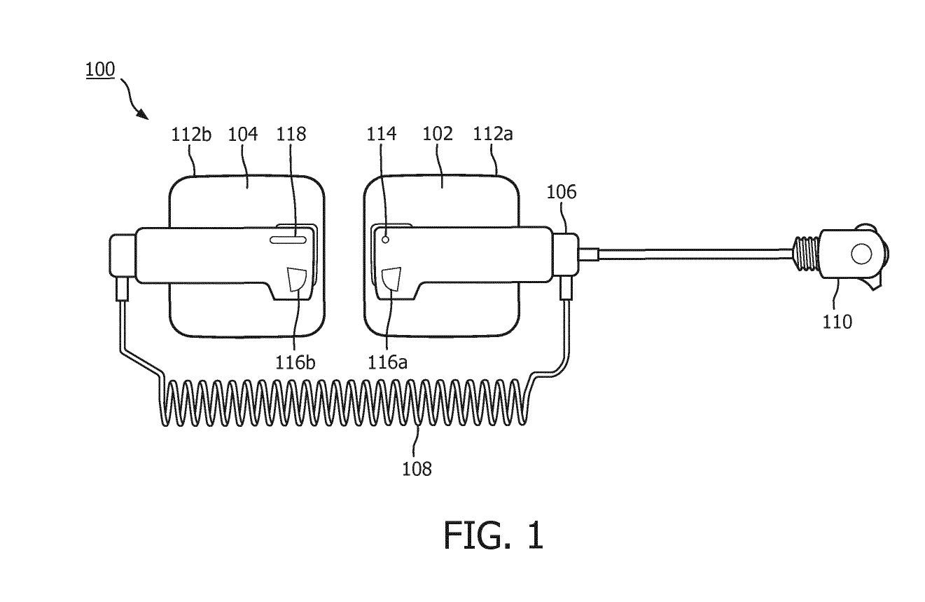

In a preferred embodiment of the present disclosure as illustrated in FIG. 1, the defibrillator paddle assembly 100 comprises an apex paddle 102 and a sternum paddle 104 connected through a node 106 located in the apex paddle 102 via a wire connector 108, and a quick connect connector 110 of the defibrillator paddle assembly 100 for connecting to or disconnecting from the defibrillator main unit (not shown). Both the apex paddle 102 and the sternum paddle 104 have electrodes 112a and 112b, infant child or adult, for delivering electric shock to a patient's body or other impedance.

The apex paddle 102 further comprises one or more sequence labels that provide instructions on the use of the defibrillator paddle assembly 100. A first sequence label is located on a charge button 114, where the charge button 114 is used to charge the defibrillator paddle assembly. A second sequence label is located on one of the at least two shock buttons 116, where the shock buttons 116 are used to deliver electric shock to the patient's body. The sternum paddle 104 further comprises a contact indicator 118, which is used to indicate the degree of contact between the patient's skin with the apex paddle 102 and the sternum paddle 104. The sternum paddle 104 also includes a sequence label which is located on another one of the at least two shock buttons 116a and 116b used to deliver electric shock into the patient's body or other impedance.

In addition to the preferred embodiment of the present disclosure illustrated in FIG. 1, the charge button 114 located in the apex paddle 102, and the shock buttons 116a and 116b on both the apex paddle 102 and the sternum paddle 104 have light indicators to indicate the sequence of steps for using the defibrillator paddle assembly 100. Furthermore, the contact indicator 118 is preferably located on the upper left corner of the sternum paddle 104 whereby a view of a user will not be blocked while activating a shock. The contact indicator 118 shows light intensity change to indicate the degree of contact between the patient's skin with the apex paddle 102 and the sternum paddle 104 ranging from a “low” state to a “high” state or degree of contact. A dimmed light indicates a poor state of contact whereas a bright light indicates a good state of contact. The contract indicator 118 may also show different colors to indicate the degree of contact between the patient's skin with the apex paddle 102 and the sternum paddle 104 ranging from a red color indicating “low” state to a green color indicating a “high” state or degree of contact.

In an alternative embodiment, the contact indicator 118 may be located on an upper right corner of the apex paddle 102, and the charge button 114 may be located on the sternum paddle 104. Concurrently, a contact indicator 118 may be located on both the apex paddle 102 and the sternum paddle 104, and a charge button 114 may be located on both the apex paddle 102 and the sternum paddle 104.

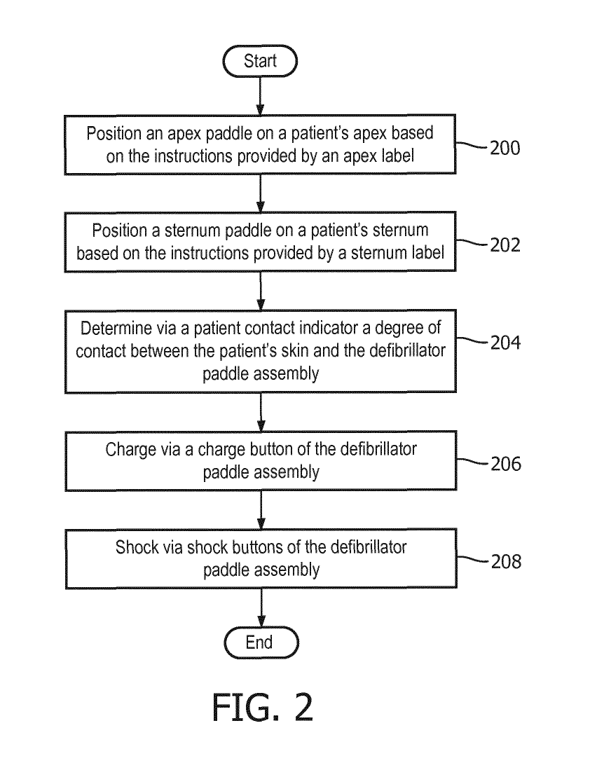

FIG. 2 illustrates a preferred method of the present disclosure. In case of emergency, for example, involving a patient suffering from cardiac arrest, a user positions an apex paddle (e.g., apex paddle 102 of FIG. 1) on the patient's apex based on the instructions provided by an apex label (step 200). Afterwards, the user positions a sternum paddle (e.g., sternum paddle 104 of FIG. 1) on the patient's sternum based on the instructions provided by a sternum label (step 202). Alternatively in practice, the apex paddle may be positioned on the patient's sternum (step 200) and the sternum paddle may be positioned on the patient's apex (step 202).

The degree of contact, signaled via a contact indicator (e.g., contact indicator 118 of FIG. 1), is determined by measuring the degree of contact between the patient's skin and the apex and sternum paddles (step 204). The contact indicator preferably has a light indicator such as an LED with varying light intensities showing a dimmed light for “low” state or poor paddle-to-skin contact, or a bright light for “high” state or good paddle-to-skin contact. When the contact indicator indicates a “high” state, the user presses the charge button of the apex paddle to charge the defibrillator paddle assembly (step 206). After charging, the user then simultaneously presses the shock buttons located in both the apex paddle and the sternum paddle to deliver the necessary electric shock through the patient's heart to reestablish the heart's normal contractions (step 208). Pressing the shock buttons simultaneously on both the apex paddle and the sternum paddle serves as a safety mechanism to prevent accidental shock to the patient. The electric shock does not activate even if the user accidentally presses one of the two shock buttons, thus preventing injuries to a patient such as burns caused by the defibrillator.

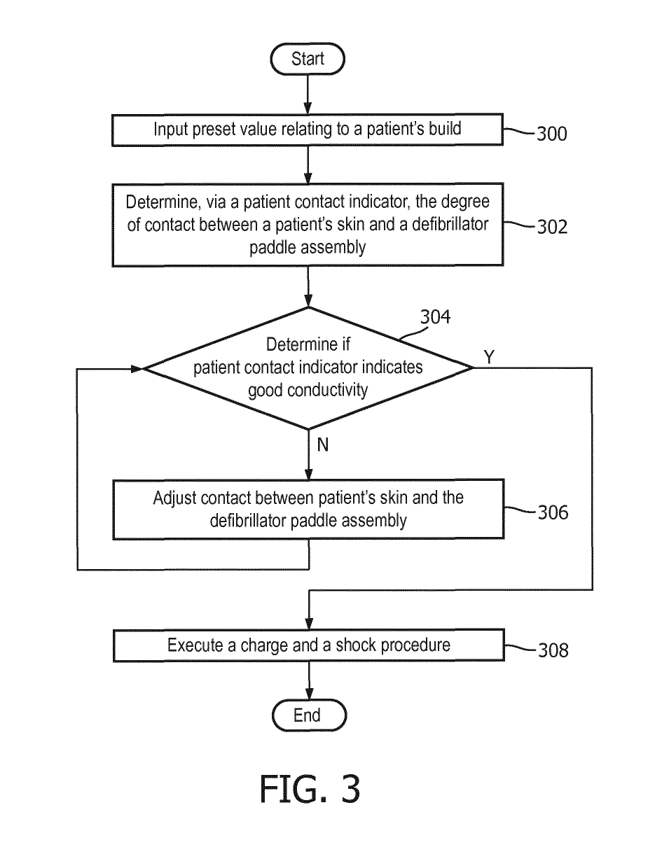

FIG. 3 illustrates a method of the exemplary embodiment of the present disclosure. A user confirms the defibrillator paddle is preset to a value based on a patient's build, adult or infant/child (step 300). A contact indicator determines the degree of contact between the patient's skin and a defibrillator paddle assembly (step 302). The contact indicator indicates a “low” state or a “high” state of contact using light intensity variations (step 304). If the contact indicator indicates a “low” state degree of contact, the user repositions the defibrillator paddle assembly and/or applies special conductive gel to the electrodes to obtain sufficient conduction between the patient's skin and the defibrillator paddle assembly (step 306). Once the contact indicator indicates a “high” state or degree of contact (step 304), the user executes the charge and shock steps involving the defibrillator paddle assembly (step 310).

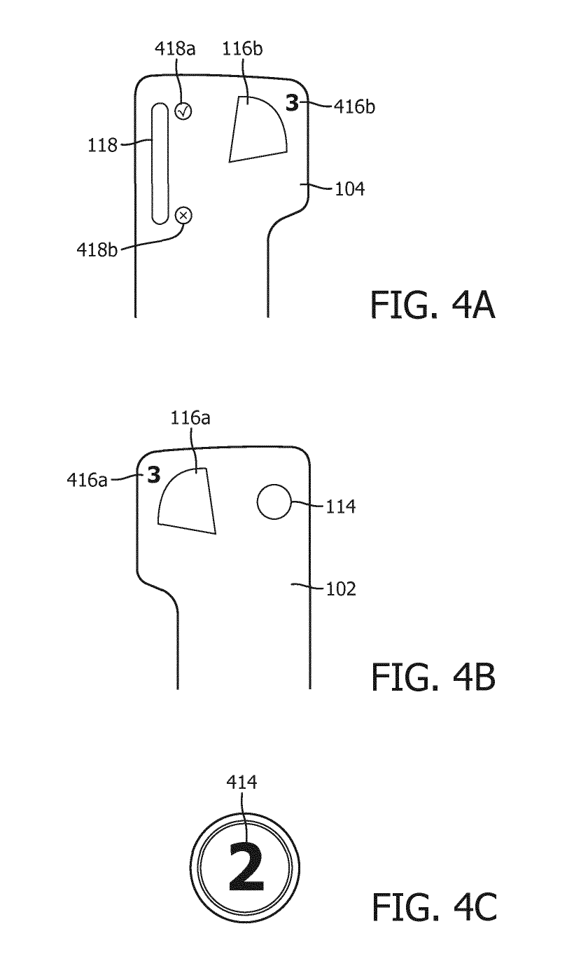

FIGS. 4A-4C illustrates an exemplary embodiment of the present disclosure. FIG. 4A shows the sternum paddle 104 with a “3” sequence label 416b, preferably of a “PHILIPS 10713” color. The contact indicator 118 includes one (1) or more LEDs (preferably ten (10) LEDs) and further includes a “high” state label 418 a, preferably of “PANTONE 354 C GREEN” color and a “low” state label, preferably of a “PANTONE 179 C RED” color. In a further embodiment of the present disclosure shown in FIG. 4B, the apex paddle 102 has a “3” sequence label 416a, preferably of a “PHILIPS 10713 DARK GREY”. Also shown in FIG. 4A is the shock button 116b on the sternum paddle 104. Also shown in FIG. 4B is the charge button 114 and the shock button 116a on the apex paddle 102. FIG. 4C, on the other hand, shows a “2” sequence labels 414, preferably of a “PHILIPS 10713 DARK GREY” and placed on charge button 114.

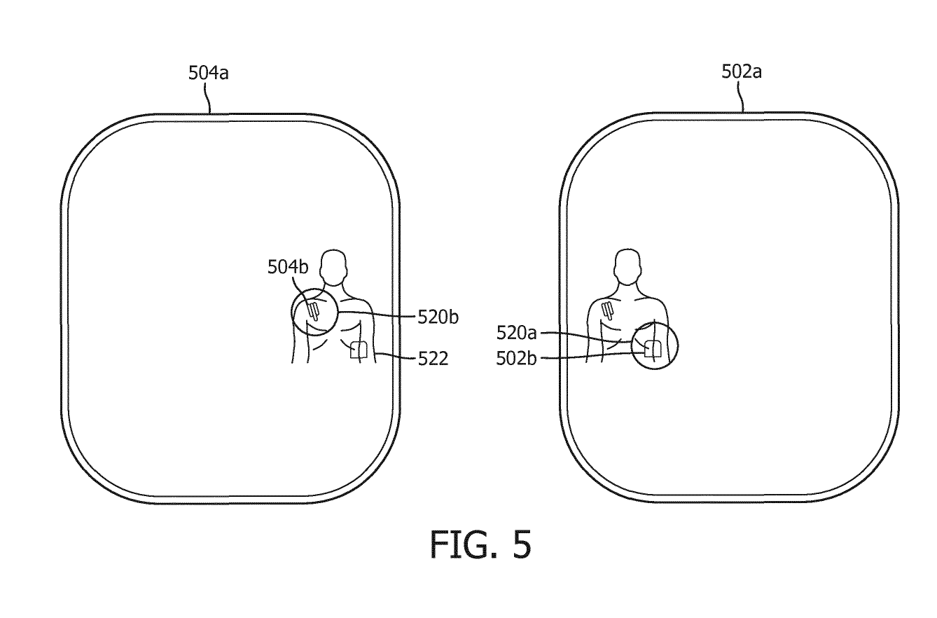

FIG. 5 illustrates another exemplary embodiment of the present disclosure. An apex label 502a and a sternum label 504a indicate the preferred positions of the apex paddle and the sternum paddle on a patient's body. An image 502b located on the apex label 502a shows the preferred position or location of the apex paddle on the patient's apex. Also, an image 504b located on the sternum label 504a shows the preferred location of the sternum paddle on the patient's sternum. In addition, in FIG. 5, image 520a preferably has a “PANTONE 179 C RED” color, image 520b preferably has a “PANTONE 179 C RED” color, and image 522 preferably has a “PHILIPS 10713 DARK GREY” color.

In another embodiment of the present disclosure, the contact indicator comprises multiple LED lights that when activated emit light intensities that vary depending on the degree of contact between the patient's skin and the defibrillator paddle assembly. For example, the contact indicator is composed of ten (10) LEDS include three (3) red LEDS, three (3) yellow LEDS and four (4) green LEDS. In this case, having only one (1) red LED activated from the group of ten (10) LEDS can be set to correspond to a “low” contact state. On the other hand, a contact indicator having all ten (10) LEDS activated can be chosen to correspond to a “high” state of contact between the patient's skin and the defibrillator paddle assembly. In another embodiment of the present disclosure, the apex and sternum paddles each has a contact indicator for showing the degree of contact between the patient's skin and the defibrillator paddles. With the apex and sternum paddles having their own contact indicators, the user will thus be able to make the necessary adjustments to either or both of the paddles to ensure proper application of the defibrillator on the patient.

A highly preferred embodiment of the present disclosure provides adjustable size electrodes for the defibrillator paddle assembly to allow the use of the assembly on patients with different body types. Thus, the present disclosure provides a larger-sized adult electrode on each of the apex paddle and sternum paddle that can be converted into smaller-sized infant/child electrode by, for example, disconnecting from a supplemental electrode from the smaller-sized infant/child electrode. Thus, if a child suddenly experiences cardiac arrest, a user can perform defibrillation with the correct electrode size by simply disconnecting the supplemental electrode from the smaller-sized electrode. If a patient is an adult with normal build, the electrodes can be converted or reconverted to the larger-sized adult electrode by simply connecting or reconnecting the supplemental electrode to the smaller-sized electrode.

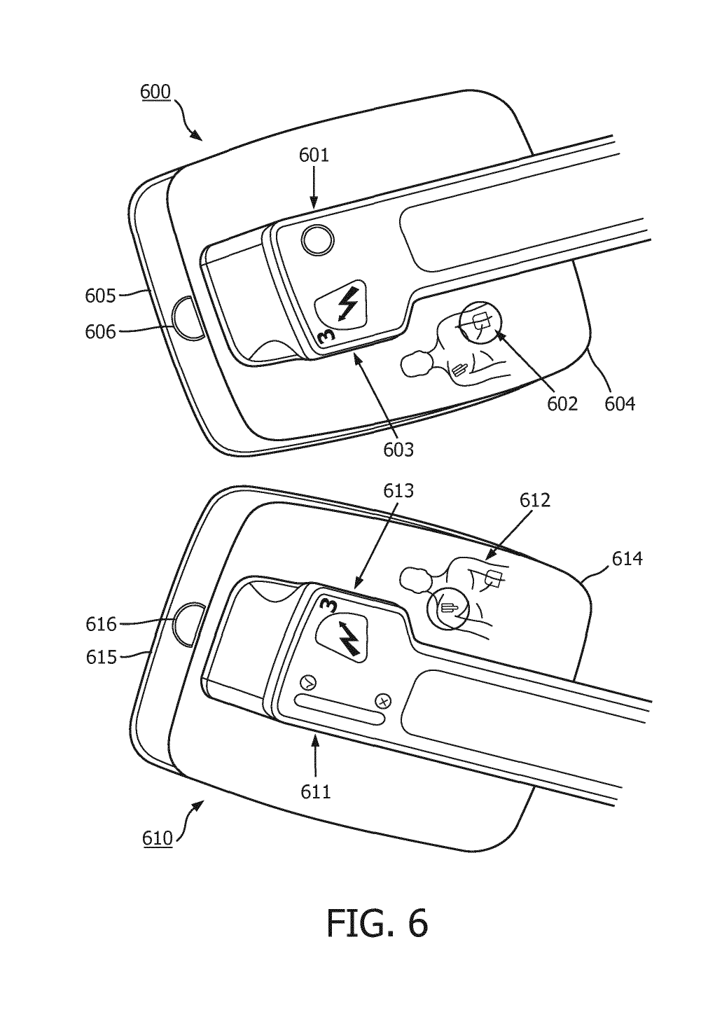

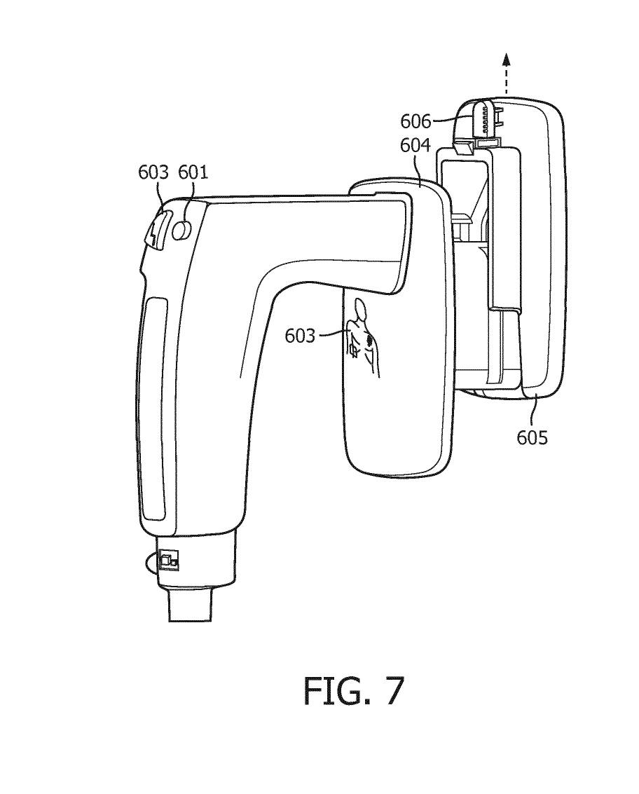

FIG. 6 shows an apex paddle 600 having a charge button 601, a proper apex paddle placement icon 602, a shock button 603, an infant/child electrode 604 and an adult electrode 605. As shown in FIG. 6, infant/child electrode 604 and adult electrode 605 are connected via a spring connector 606. FIG. 7 shows an exemplary activation of the spring connector 606 to enable a disconnection of the adult electrode 605 from the infant/child electrode 604 as symbolized by the arrow to thereby provide an exclusive use of the infant/child electrode 604.

FIG. 6 further shows a sternum paddle 610 having a contact indicator 611, a proper sternum paddle placement icon 612, a shock button 613, an infant/child electrode 614 and a supplemental electrode 615. As shown in FIG. 6, infant/child electrode 614 and an adult electrode 615 are connected via a spring connector 616. Similar to the apex paddle 600 as shown in FIG. 7, spring connector 616 may be activated to enable a disconnection of the adult electrode 615 from the infant/child electrode 614 to thereby provide an exclusive use of the infant/child electrode 614.

In another embodiment of the present disclosure, an additional safety measure is implemented for preventing accidental shock to a medical personnel. In a high-humidity environment, accidental electric shock may be imparted not only to the patient but also to the medical personnel or other persons in proximity to the patient. Incorporating a moisture sensor in the defibrillator paddle assembly of the present disclosure can minimize the possibility of unintended electrical shocks to non-patients. Preferably, the moisture sensor sends a signal to the deactivator to deactivate the charge and shock buttons of the defibrillator paddle assembly when the surrounding's humidity or moisture level exceeds a preset threshold. Preferably, a moisture sensor is installed in at least one of the two defibrillator paddles.

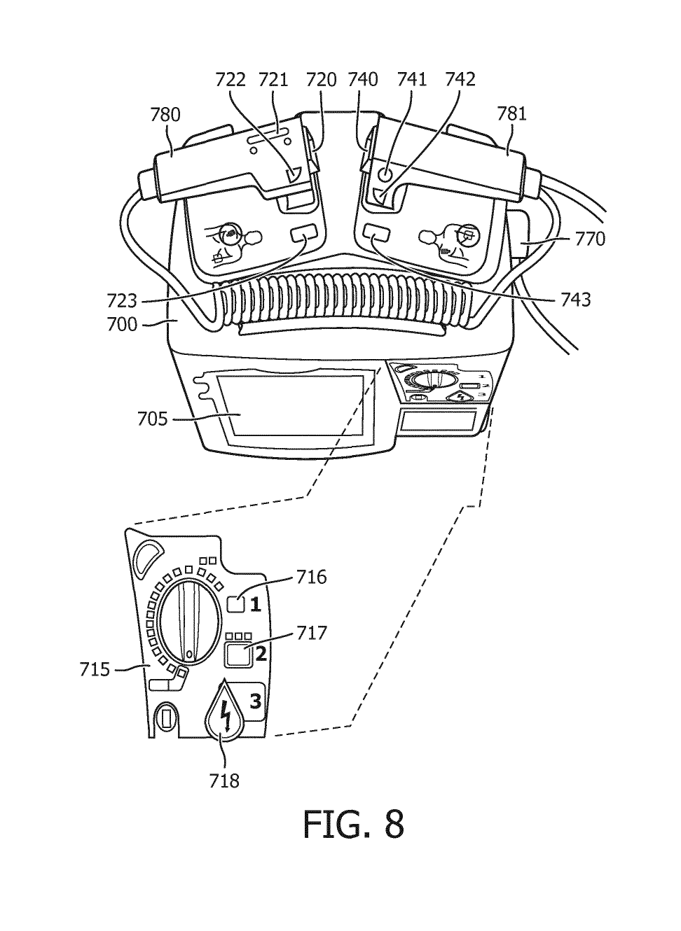

FIG. 8 shows a defibrillator 700 having a sternum paddle 780 and an apex paddle 781. A base of defibrillator 700 includes a connector port 770 for paddles 780 and 781, and a user control panel 715. Messages regarding a status of defibrillator 700, including a connection of paddles 780 and 781 thereto, may displayed on optional display 705.

User control panel 715 includes graphic and text labeling indicia that are located adjacent to the controls associated with charging and discharging the defibrillating high energy circuit. More particularly, FIG. 8 illustrates the use of text indications located next to the respective control, such as “Select Energy” 716, “Charge” 717, or “Shock” 718. Alternatively, a 1-2-3 numbering scheme may be shown on user control panel 715 to help guide the user in the proper execution of the steps of a rescue. Thus, the first step of selecting energy may be indicated by a “1” label, the second step of charging the high energy circuit may be indicated by a “2” label, and the third step of delivering the shock may be indicated by a “3” label, each appearing adjacent the respective control.

The sternum paddle 780 and the apex paddle 781 are employed by pressing the respective electrodes to the patient's chest at the standard anterior-anterior (A-A) positions. An internal defibrillator high energy circuit (not shown) is charged by pressing charge button 741. When the defibrillator 700 is fully charged and ready to shock, shock buttons 722 and 742 will flash. Then, when good electrical contact with the patient is established as indicated by contact indicator 721, the user presses both shock buttons 722 and 742 simultaneously to deliver the therapy. As an additional safety measure, the sternum paddle 780 may include a moisture sensor (not shown) and/or the apex paddle 781 may include a moisture sensor (not shown).

The present disclosure is not intended to be restricted to the several exemplary embodiments of the invention described above. Other variations that may be envisioned by those skilled in the art are intended to fall within the disclosure.

1. A defibrillator paddle assembly, comprising:
an apex paddle;
a sternum paddle; and
at least one visual contact indicator configured to indicate at least one of a degree of impedance of the apex paddle and a degree of impedance of the sternum paddle, wherein the at least one visual contact indicator is located within the defibrillator paddle assembly relative to one of the apex paddle and the sternum paddle.

2. The defibrillator paddle assembly of claim 1,
wherein the degree of impedance of the apex paddle represents a degree of contact between the apex paddle and a patient's apex; and
wherein the degree of impedance of the sternum paddle represents a degree of contact between the sternum paddle and a patient's sternum.

3. The defibrillator paddle assembly of claim 1,
wherein the at least one visual contact indicator is located on the apex paddle.

4. The defibrillator paddle assembly of claim 3,
wherein the at least one visual contact indicator includes a light-emitting assembly configured to emit a variable light intensity indicative of the degree of impedance of the apex paddle.

5. The defibrillator paddle assembly of claim 4,
wherein the variable light intensity of the light-emitting assembly is further indicative of the degree of impedance of the sternum paddle.

6. The defibrillator paddle assembly of claim 1,
wherein the at least one visual contact indicator is located on the sternum paddle.

7. The defibrillator paddle assembly of claim 6,
wherein the at least one contact indicator includes a light-emitting assembly configured to emit a variable light intensity indicative of the degree of impedance of the sternum paddle.

8. The defibrillator paddle assembly of claim 7,
wherein the variable light intensity of the light-emitting assembly is further indicative of the degree of impedance of the apex paddle.

9. The defibrillator paddle assembly of claim 1,
wherein the apex paddle includes a charge button.

10. The defibrillator paddle assembly of claim 1,
wherein the sternum paddle includes a charge button.

11. The defibrillator paddle assembly of claim 1,
wherein the apex paddle includes an apex label with an instruction to place the apex paddle on the patient's apex.

12. The defibrillator paddle assembly of claim 1,
wherein the sternum paddle includes an apex label with an instruction to place the sternum paddle on the patient's sternum.

13. The defibrillator paddle assembly of claim 1, wherein the apex paddle includes:
an infant/child electrode; and
a supplement electrode connectable to the infant/child electrode to construct an adult electrode.

14. The defibrillator paddle assembly of claim 1, wherein the sternum paddle includes:
an infant/child electrode; and
a supplement electrode connectable to the infant/child electrode to construct an adult electrode.

15. The defibrillator paddle assembly of claim 1,
wherein the apex paddle includes an apex shock button; and
wherein the sternum paddle includes a sternum shock button.

16. A method for enabling a defibrillator paddle assembly including an apex paddle and a sternum paddle used in patient's cardiac arrest treatment, the method comprising:
positioning the apex paddle on a patient's apex;
positioning the sternum paddle on a patient's sternum; and
determining, via a contact indicator of the defibrillator paddle assembly, a degree of contact between the patient's skin and the defibrillator paddle assembly.

17. The method of claim 16,
wherein the apex paddle is positioned on the patient's apex based on an instruction provided by the apex paddle; and
wherein the sternum paddle is positioned on the patient's sternum based on an instruction provided by the sternum paddle.

18. The method of claim 16, further comprising:
activating a charge button and one or more shock buttons of the defibrillator paddle assembly responsive to an indication of a high state by the contact indicator;
charging, via the activated charge button, the defibrillator paddle assembly; and
delivering a shock, via the one or more activated shock buttons, on the patient's body for treating the patient's cardiac arrest.

19. The method of claim 16, wherein, prior to the positioning the apex paddle on the patient's apex and the positioning the sternum paddle on the patient's sternum, for each of the apex paddle and the sternum paddle, an adult electrode is connected to an infant/child electrode.

20. The method of claim 16, wherein, prior to the positioning the apex paddle on the patient's apex and the positioning the sternum paddle on the patient's sternum, for each of the apex paddle and the sternum paddle, an adult electrode is disconnected from an infant/child electrode.
